(19) United States
(12) Patent Application Publication
(10) Pub. No.: US 2008/0050449 A1
(43) Pub. Date: Feb. 28, 2008

(54) Controlled release formulation of tolterodine

(76) Inventors: Dafna Arieli, Kadima (IL); Tomer Gold, Ra'anana (IL)

Correspondence Address: KENYON & KENYON LLP, ONE BROADWAY, NEW YORK, NY, 10004, US

(21) Appl. No.: 11/726,535

(22) Filed: Mar. 21, 2007

Related U.S. Application Data
(60) Provisional application No. 60/784,573, filed on Mar. 21, 2006.

(51) Int. Cl. A61K 31/135; A61K 9/14

(52) U.S. Cl. 424/494; 424/489; 424/490; 424/497; 514/648

(57) ABSTRACT

The invention encompasses stable multiparticulate pharmaceutical compositions of tolterodine having at least one pharmaceutically acceptable excipient and at least two populations of multiparticulates each population having tolterodine or a salt thereof and the ratio of the populations is from 90:10 to 10:90 by weight, wherein after storage for 1 month at 40° C. and 75% relative humidity the difference between the dissolution profile at 4 hours is no more than about 5% when compared to the dissolution profile at the time of manufacture.

### RELATED APPLICATIONS

This application claims the benefit of U.S. provisional Application Ser. No. 60/784,573, filed Mar. 21, 2006, hereby incorporated by reference.

### FIELD OF THE INVENTION

The invention encompasses controlled release tolterodine formulations and methods for preparing thereof.

### BACKGROUND OF THE INVENTION

One of the recently found muscarinic receptor antagonists is tolterodine, (R)-N,N-diisopropyl-3-(2-hydroxy-5-methylphenyl)-3-phenylpropanamine. Both tolterodine and its major metabolite, the 5-hydroxy derivative and their pharmaceutically acceptable salts appear to be active. An important compound of tolterodine is its L-tartrate salt form. Tolterodine's chemical structure is shown below.

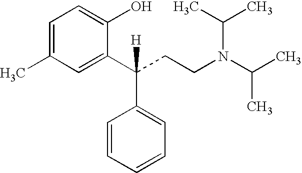

Tolterodine L-Tartrate is presently being sold in a number of different countries under the name Detrol® or Detrositol® as marketed by Pharmacia (now part of Pfizer). U.S. Pat. No. 6,911,217 disclosed one formulation of tolterodine and it is understood that the formulation is composed of three-layered coated multiparticulates. The multiparticulates include: (i) a core unit of water-swellable or water-insoluble inert material; (ii) a first layer on the core of a substantially water-insoluble polymer; (iii) a second layer covering the first layer and containing the active ingredient; and (iv) a third layer of polymer on the second layer effective for controlled release of the active ingredient. The first layer supposedly is adapted to control water penetration into the core.

U.S. Pat. No. 6,911,217 discloses a process for preparing such controlled release multiparticulates. The second layer is applied from a diluted solution of the active ingredient and a binder; it is believed that this may require a relatively long coating process. Moreover, the third layer is an aqueous dispersion of a hydrophobic polymer and this application typically requires an additional curing step, which may further lengthen the production process.

In the examples of U.S. Pat. No. 6,911,217, the aqueous ethylcellulose dispersion applied for both the first and third layer is Surelease®. Surelease® is a commercial dispersion that contains oleic acid as a stabilizer in ammoniated water. Thus, in this process ammonium oleate may form and may be present in the final coating which may result in unwanted migration/complexation of the active ingredient. Such interactions may dominate under long curing periods at elevated temperatures.

PCT publication WO 04/105735 discloses a controlled release pharmaceutical composition of tolterodine that includes one or more coated units. Each unit has a core, a first layer and a second layer. The first layer surrounds the core and includes tolterodine and one or more hydrophilic polymers. The second layer includes one or more polymers effective for controlled release of the tolterodine from the first layer.

Controlled release dosage forms need to have consistent drug release between dose units prepared in different production batches and throughout the shelf life of the finished product. Such release stability requirements are set forth in the Good Manufacturing Practices (GMPs), The United State Pharmacopoeia (USP), in New Drug Applications (NDAs) and Investigational New Drug applications (INDs).

U.S. Publication No. 2003/152,624 states that it was “unexpectedly discovered that compositions described in International Patent Publication No. WO 00/27364 can exhibit undesirable drug release variability.” Both U.S. Publication No. 2003/152,624 and WO 00/27364 are assigned to Pharmacia Co. Furthermore, U.S. Pat. No. 6,911,217 is one of the corresponding U.S. patents which issued from WO 00/27364.

U.S. Publication No. 2003/152,624 discloses a controlled release dosage form having improved drug release properties. The controlled release formulation includes dosage units of tolterodine or a tolterodine-related compound as an active drug and a pharmaceutically acceptable polymer-based release-controlling component having a particular age distribution at the time of manufacture. The age distribution of the release-controlling component at time of manufacturing the dosage units is such that upon randomly sampling a plurality of dosage units and individually testing the in vitro dissolution of each one, the drug release after 3 hours varies by not more than 15% of a target.

In light of the above background, it will be appreciated by those versed in the pharmaceutical arts that a need exists for a controlled release pharmaceutical composition that can deliver tolterodine in a controlled, extended dose. At the same time, the composition must be governed by shelf life stability, and formulated by employing a simple process that involves shorter and/or fewer layering steps. Clearly, the use of polymer aqueous (water) dispersions, which may enhance unwanted drug release variability, should be avoided.

### SUMMARY OF THE INVENTION

One embodiment of the invention encompasses a stable multiparticulate pharmaceutical composition of tolterodine comprising at least one pharmaceutically acceptable excipient and at least two populations of multiparticulates each population having tolterodine or a salt thereof wherein after a storage for 1 month at 40° C. and 75% relative humidity, the difference between the dissolution profile of the pharmaceutical composition at 4 hours is no more than about 5%, when compared with the dissolution profile at the time of manufacture. The populations may comprise a first population of multiparticulates having a water soluble sphere core and a second population of multiparticulates having a nonsoluble and/or swellable sphere core.

In the stable multiparticulate pharmaceutical formulation, the ratio of the two populations is about 90:10 to 90:10 by weight, and preferably, the ratio is about 20:80 to 80:20 by weight. The core may be sphere shaped and has a diameter of about 0.3 mm to about 1 mm, and preferably, the core is sphere shaped and has a diameter of about 0.4 mm to about 0.8 mm.

The water soluble sphere core is preferably a sugar sphere core. The nonsoluble and/or swellable sphere core is preferably a cellulose sphere core. In the formulation, the ratio of sugar sphere cores to cellulose sphere core may be in a ratio of 1:1 to 2:1 by weight.

In the first or second population of multiparticulates each particulate has a core with a drug containing layer and a control release layer; wherein the drug containing layer surrounds the core and has i) tolterodine and/or a metabolite or pharmaceutically acceptable salt or salts thereof and ii) at least one hydrophilic polymer binder; and the control release layer surrounds the drug containing layer and has at least one extended release material and at least one release modifying material. Preferably, the tolterodine salt is tolterodine L-tartrate.

The stable multiparticulate pharmaceutical formulation may further comprise a water soluble polymer coating between the core and the drug containing layer. Optionally, the control release layer further comprises a plasticizer. The ratio of tolterodine to hydrophilic polymer binder may be about 1:2 to 5:1 by weight. The tolterodine may be micronized and have a particle size distribution wherein the d(0.9) value is less than or equal to about 80 microns, and preferably the tolterodine has a particle size distribution wherein the d(0.9) value is less than or equal to about 50 microns.

In the stable multiparticulate pharmaceutical formulation the hydrophilic polymer binder may be polyvinyl pyrrolidone, hydroxypropylmethyl cellulose, or hydroxypropyl cellulose. The drug containing layer comprises about 1% to about 10% by weight of the final particulate. The ratio of the extended release material to the release modifying material is from about 6:1 to about 1.5:1 by weight. The extended release material may be ethylcellulose or polymethacrylate polymer, such as ethylcellulose having a viscosity of about 7 cPs to about 50 cPs.

The release modifying material may be low viscosity hydroxypropyl methylcellulose, such as hydroxypropyl methylcellulose having a viscosity of about 3 cPs to about 6 cPs.

In the stable multiparticulate pharmaceutical formulation the ratio of the extended release material and the release modifying material to plasticizer may be about 25:1 to 10:1 by weight. In the stable multiparticulate pharmaceutical formulation the control release layer may comprise about 4% to about 30% by weight of the final multiparticulate and preferably, comprises about 6% to about 25% by weight of the final multiparticulate.

Another embodiment of the invention encompasses a process for preparing a stable multiparticulate pharmaceutical composition of tolterodine comprising: mixing at least one pharmaceutically acceptable excipient and at least two populations of multiparticulates, each population having tolterodine or a salt thereof and each population having a tolterodine dissolution profile taken at 4 hours which fluctuates greater than about 5% after storage for one month at 40° C. and 75% relative humidity when compared to the same population at the time of manufacture to obtain a stable multiparticulate pharmaceutical composition wherein after storage for 1 month at 40° C. and 75% relative humidity, the difference between the dissolution profile of the pharmaceutical composition at 4 hours is not more than about 5% when compared with the dissolution profile at the time of manufacture. The populations preferably comprise a first population of multiparticulates having a water soluble sphere core and a second population of multiparticulates having a nonsoluble and/or swellable sphere core.

In one particular embodiment, the multiparticulates are prepared by providing at least one core comprising a combination of water soluble sphere and nonsoluble and/or swellable spheres in a ratio of about 10:90 to about 90:10 by weight; applying to the core a drug containing material in an amount sufficient to form a drug containing layer to form a core with a drug containing layer; and applying to the drug containing layer a control release material in an amount sufficient to form a control release layer; wherein the drug containing material comprises i) a drug which is at least one antimuscarinic antagonist and ii) at least one hydrophilic polymer binder; and the control release material comprises at least one extended release polymer and at least one release modifying polymer. Optionally, the process includes a drying step after applying the drug containing material to the core. Optionally, the process includes a drying step after applying the control release material.

The first applying step may comprise charging the core into a fluidized bed device equipped with a Wurster column and applying a coating of the drug containing material to form the drug containing layer. The drug containing material may be a dispersion prepared by dissolving the hydrophilic polymer binder in purified water to form a solution and then mixing the solution with tolterodine to form a homogeneous dispersion.

The second applying step may comprise charging the core with a drug containing layer into a Wurster fluid bed and applying a control release material of at least one extended release polymer and at least one release modifying polymer. The control release material may be prepared by mixing two separate solutions of a first solution of ethylcellulose dissolved in ethanol and a second solution of hydroxypropylmethyl cellulose dissolved in purified water. The control release material may be prepared by mixing hydroxypropyl methylcellulose and ethylcellulose in ethanol.

Yet another embodiment of the invention encompasses a multiparticulate pharmaceutical composition of tolterodine having a reproducible dissolution profile comprising a combination of at least a first and a second population of multiparticulates having tolterodine and at least one pharmaceutically acceptable excipient, wherein the dissolution profile of a first batch or lot of the pharmaceutical composition that is measured at 4 hours differs by no more than about 5% from the dissolution profile of other lots or batches of the pharmaceutical composition.

One embodiment of the invention encompasses a process for preparing a multiparticulate pharmaceutical composition of tolterodine having a reproducible dissolution profile comprising: a) preparing at least two populations of multiparticulates having different dissolution profiles, wherein the difference is more than about 5% at 4 hours of dissolution and no more than about 10% at 12 hours of dissolution; b) characterizing the dissolution profiles of each population; and c) mixing a weighted portion of each population to obtain a pharmaceutical composition having a reproducible dissolution profile. Another embodiment of the invention encompasses a method of preparing a tolterodine formulation comprising combining a plurality of tolterodine containing multiparticulates into a dosage unit.

### DESCRIPTION OF THE FIGURES

FIG. 1 illustrates in vitro dissolution profiles of tolterodine tartrate formulations of Examples 1-3, where the core composition was varied.

FIG. 2 illustrates in vitro dissolution profiles of tolterodine tartrate formulations of Examples 4-6, where the composition of the drug containing layer was varied.

FIG. 3a illustrates in vitro dissolution profiles of tolterodine tartrate formulations of Examples 7-9, where the relative amount of release modifying polymer in a hydroalcoholic solution was varied.

FIG. 3b illustrates in vitro dissolution profiles of tolterodine tartrate formulations of Examples 10-12, where the relative amount of release modifying polymer in alcoholic solution was varied.

FIG. 4 illustrates in vitro dissolution profiles of tolterodine tartrate formulations of Example 13a and 13b using different process solvents for applying the extended release layer.

FIG. 5 illustrates in vitro dissolution profiles of tolterodine tartrate formulations of Examples 14 and 15 using HPMC of different viscosities.

FIG. 6 illustrates in vitro dissolution profiles of tolterodine tartrate formulations of Examples 16 and 17 using different viscosities.

FIGS. 7, 8, and 9 illustrate in vitro dissolution profiles of tolterodine tartrate formulations having different core materials after different storage conditions (Examples 18,19, and 20).

FIGS. 10 and 11 illustrate the effect of micronizing tolterodine tartrate on in vitro dissolution profiles after different storage conditions (Examples 21-22).

FIG. 12 illustrates the effect of micronizing tolterodine tartrate and core materials on in vitro dissolution profiles after different storage conditions (Example 8a,b).

### DETAILED DESCRIPTION OF THE INVENTION

The invention encompasses multiparticulate pharmaceutical formulations of tolterodine which are stable and/or have a reproducible dissolution profile and methods of making the same. The rate of drug release can be controlled by altering various formulation parameters such as material selection. For example, the judicious selection of core material, hydrophilic polymeric binder in the first layer, and/or the selection of the extended release polymer or release modifying polymer in the second layer can be used to control the drug release rate and the dissolution profile of multiparticulate populations. For example, the invention yields multiparticulate pharmaceutical compositions with a reproducible dissolution profile that can deliver a particular drug over an extended period of time by using at least two populations of multiparticulates with different dissolution profiles. Also, the invention encompasses stable multiparticulate pharmaceutical compositions of tolterodine comprising at least two populations of multiparticulates having a non-stable dissolution profile.

As used herein when referring to a formulation, the term “stable dissolution profile” means that after storage for 1 month at 40° C. and 75% relative humidity, the difference between the dissolution profile of a pharmaceutical composition at 4 hours is not more than about 5% when compared to the dissolution profile at the time of manufacture.

As used herein when referring to a formulation, the term “unstable dissolution profile” means that when comparing the dissolution profile taken at 4 hours of a pharmaceutical formulation that was stored for one month at 40° C. and 75% relative humidity to the dissolution profile of the same formulation taken at the time of manufacture the difference is more than about 5%.

As used herein when referring to a formulation, the term “reproducible dissolution profile” means that the difference between the dissolution profile of a first batch or lot measured at 4 hours and the dissolution profile of other lots or batches of the pharmaceutical formulation is not more than 5%.

As used herein, the term “time of manufacture” means the time when a skilled artisan would consider the manufacture of the pharmaceutical formulation to be complete and ready for use as determined in the normal course of business. This term may also be referred as “time zero.”

The dissolution profiles discussed above are determined by dissolving the formulation or multiparticulate population at 37° C. in 0.05 M Phosphate buffer with a pH 6.8 using a USP dissolution test apparatus I (basket) and using either 800 ml of solution (when testing over 0-12 hours) or 900 ml of solution (when testing over 0-14 hours).

The stable multiparticulate pharmaceutical composition of tolterodine comprises at least two populations of multiparticulates each population having tolterodine or a salt thereof and each population having an unstable dissolution profile.

In particular, the invention encompasses a stable formulation of tolterodine L-tartrate comprising multiparticulates having a core and two or more coatings and methods of preparing the same. Each multiparticulate includes at least a core, a first layer (tolterodine containing layer), and a second layer (control release layer). The formulations of the invention overcome the above-described shelf-life in-vitro dissolution stability problems.

In one embodiment, the first layer, the tolterodine containing layer, surrounding the core includes tolterodine and a hydrophilic polymer binder. The second layer, the control release layer, surrounds the first layer and includes one or more polymers that are effective for controlling the release of tolterodine from the first layer. As used herein, unless otherwise defined, the term “surrounding” as applied to coatings refers to a coating that partially or completely surrounds a core or coated core. Optionally, the control release layer may include one or more soluble plasticizers or antitacking agents, adapted to modify the controlled drug release rate. Soluble plasticizers or antitacking agents include, but are not limited to, triethyl citrate or polyethylene glycol. Optionally, the core may be coated first with a hydrophilic polymeric layer prior to the application of the drug containing layer.

The core may be selected from any suitable material and is preferably in the shape of a sphere. Preferably, the core may is a mixture of various cores where each core or group of cores are of a different material. When the core is a combination of cores of different materials, then at least one core or “core type” is made of a water soluble material. Water penetration into the core can be controlled by combining cores of different materials that significantly differ by their water solubility parameter. Thus, for example, drug release control may be achieved by using at least one water soluble core mixed with a nonsoluble and/or swellable core at a defined ratio. The selection of core material may also provide shelf life stability to the drug as illustrated by the in vitro dissolution profile studies shown in Example 18-20.

The multiparticulate core material mainly determines the drug dissolution profile of the population of microparticulates. Hence, the drug dissolution profile of a pharmaceutical composition can be controlled by the selection of core materials used in the microparticulates and the ratio of different microparticulates populations. When using two populations of microparticulates, typically the ratio of the two populations is about 10:90 to about 90:10 by weight. Preferably, the ratio of populations is about 20:80 to 80:20 by weight, and more preferably, the ratio is about 50:50 to about 70:30 by weight.

Typically, the core is a combination of soluble spheres and nonsoluble and/or swellable spheres in a ratio of about 10:90 to about 90:10 by weight. Preferably, the ratio of soluble spheres to nonsoluble and/or swellable spheres is about 20:80 to 80:20 by weight, and more preferably, the ratio is about 50:50 to about 70:30 by weight. The resulting core may be sphere shaped and has a diameter of about 0.3 mm to about 1 mm. Preferably, the diameter is about 0.4 mm to about 0.8 mm, and more preferably, the diameter is about 0.5 mm to about 0.7 mm.

The core may be made of sugar spheres and microcrystalline cellulose spheres. The dissolution of a coated sugar core decreases over time; thus, the longer the coated core is stored, over time a reduced amount of drug is delivered (Example 18). The microcrystalline cellulose core, because it is a different material, has a different drug release profile than that of a sugar core. The dissolution of a coated microcrystalline cellulose core increases over time; thus, the longer the coated core is stored, over time an increased amount of drug is delivered (Example 19).

Preferably, the ratio of sugar spheres to microcrystalline cellulose spheres is about 1:1 to 2:1 by weight. Commercially available sugar spheres include Suglets® sold by NP Pharma (Bazainville, France) and commercially available microcrystalline cellulose spheres include Cellets® sold by Syntapharm (Germany). When the core is a combination of sugar spheres and microcrystalline cellulose spheres, the weight of the cores is about 70% to about 90% by weight of the final multiparticulates.

The core may be optionally coated with the hydrophilic polymeric layer comprising a soluble polymer and a soluble plasticizer or antitacking agent. Soluble polymers include, but are not limited to, cellulose derivatives, such as hydroxypropylmethyl cellulose. Soluble plasticizers or antitacking agents are as described previously.

The first layer, the drug containing layer, is a combination of at least one antimuscarinic antagonist, such as tolterodine or its acceptable pharmaceutical salt, and at least one hydrophilic polymer binder. Typically, the ratio of drug to hydrophilic polymer binder should be sufficient to effectively bind the drug to the core, such that a collection of multiparticulates can deliver a therapeutically effective amount of the drug, such as tolterodine. The first layer may allow for control of the rate of drug release. The water solubility of the hydrophilic polymer binder used in the first layer may be a critical parameter used to control the drug release rate. The drug dissolution profile can be raised or decreased by modifying the water solubility of the hydrophilic polymer binder as well as its absolute amount. Hydrophilic polymer binders include, but are not limited to, polyvinyl pyrrolidone, or cellulose derivatives. Cellulose derivatives include hydroxypropyl cellulose and hydroxypropylmethyl cellulose, preferably, the hydroxypropyl cellulose with a viscosity of 100 cPs. The drug containing layer may optionally include a soluble plasticizer or antitacking agent as described previously.

The size of the core and the number of multiparticulates in one unit dose will determine the thickness of the drug containing layer. For example, if the number of multiparticulates is kept constant, a smaller core will require a thicker layer to deliver the same amount of drug included in a larger core with a thinner layer. The first layer comprises about 1% to about 10% by weight of the final multiparticulate. Preferably, the first layer comprises about 1% to about 7% by weight, and more preferably, about 2% to about 5% by weight of the final multiparticulate. In one embodiment, the ratio of tolterodine to hydrophilic polymer is about 1:2 to 5:1 by weight. Tolterodine may be in the form of a salt including, but not limited to, L-tartrate tolterodine. The L-tartrate tolterodine may be micronized. If micronized, the L-tartrate tolterodine should have a particle size d(0.9) value of less than about 80 microns, and preferably, not more than (NMT) about 50 microns. More preferably, the particle size is d(0.9) NMT about 25 microns.

Drug release may be controlled by the selection of the ratio of extended release material to release modifying material in the second layer, the control release layer. If a hydroalcoholic solution is chosen for the control release layer, then the drug release dissolution profile can be varied from an immediate release profile to a controlled release profile by careful selection of the ratio of materials. However, if a similar control release formulation is prepared in 95% alcoholic solution, then the sensitivity of the dissolution profile to the above (extended- to modifying release polymer) ratio is significantly reduced. The latter can be harnessed to improve process reproducibility.

The control release layer is a combination of at least one extended release material and at least one release modifying material. The extended release material is usually a hydrophobic film forming polymer. Hydrophobic film forming polymers include, but are not limited to, ethylcellulose or polymethacrylate polymers. The release modifying material is usually a hydrophilic polymer and/or a plasticizer. Release modifying materials include, but are not limited to, low viscosity hydroxypropylmethyl cellulose or polyethylene glycol. Once the ratio of the extended release polymer to release modifying material is determined, the dissolution profile can be further modified by changing the grade (viscosity) of each of the polymers. For example, preferably, the ethylcellulose has a viscosity of about 7 cPs to about 50 cPs. Preferably, the release modifying material is hydroxypropylmethyl cellulose with a viscosity of about 3 cPs to about 6 cPs.

The ratio between extended release material and release modifying material and the optional plasticizer allows establishment of a pre-determined release rate of the drug from the coated spheres. Typically, the ratio of extended release material to release modifying material is about 6:1 to 2:1 by weight. For example in one embodiment, the ratio between extended release material, ethylcellulose, and release modifying material, HPMC, was varied between about 6:1 to about 1.5:1 by weight. However, when a plasticizer is used in the control release layer, the ratio of extended release material and release modifying material to plasticizer is about 25:1 to 10:1 by weight. The control release layer comprises about 4% to about 30% by weight of the final multiparticulate. Preferably, the second layer comprises about 6% to about 25% by weight, and more preferably about 8% to about 20% by weight of the final multiparticulate.

In addition to the formulation parameters for the control release layer, the dissolution profile can be altered by modifying the process parameters. The process parameters include the solvent system used to dissolve the two different polymers and produce the solution of the control release coating material. For example, the control release coating material may be applied as a 7.3% w/w hydro-alcoholic solution (ratio of water to alcohol is 16:84) or a 6.0% w/w alcoholic solution.

The invention also encompasses a method for preparing the stable multiparticulate pharmaceutical compositions of tolterodine. The method comprises mixing a pharmaceutically acceptable excipient with at least two populations of multiparticulates, each population having tolterodine or a salt thereof and each population having a tolterodine dissolution profile taken at 4 hours which fluctuates greater than about 5% after storage for one month at 40° C. and 75% relative humidity when compared to the same population at the time of manufacture to obtain a stable multiparticulate pharmaceutical composition, wherein after storage for one month at 40° C. and 75% relative humidity, the difference between the dissolution profile of the stable pharmaceutical composition at 4 hours is not more than about 5% when compared to the dissolution profile at the time of manufacture.

A method of preparing the multiparticulates with a two layer coating described above comprises supplying at least one core; applying a drug containing material to the core to form a drug containing layer, wherein the drug containing layer comprises at least one antimuscarinic antagonist and at least one hydrophilic polymer binder; and applying a control release material to the drug containing layer to form a control release layer, wherein the control release layer comprises at least one extended release material and at least one release modifying material.

The first layer is applied by charging the core into a fluidized bed device equipped with a Wurster column (Wurster fluid bed) and applying a coating of drug containing dispersion or solution to form a first layer, i.e., a drug containing layer. When using a Wurster column the nominal inlet air temperature is about 50° C. to 55° C. and the exhaust air temperature is about 28° C. to about 34° C., and preferably about 30° C. to about 32° C. The drug containing dispersion is prepared by dissolving the hydrophilic polymer binder, for example hydroxypropyl cellulose or hydroxypropyl methyl cellulose, in purified water, and then mixing the solution, with tolterodine to form a homogeneous dispersion. Preferably, micronized tolterodine L-tartrate is used. If necessary, after applying the first coating, the process may be followed by drying for a sufficient amount of time, for example 15 minutes. The drying may be performed in the Wurster column (Wurster-drying) at a nominal inlet air temperature of about 50° C. to 60° C. Drying may be terminated once the nominal exhaust air temperature is about 40° C.

The second layer, i.e., the control release layer, is applied by charging the coated cores into the Wurster fluid bed and applying a coating solution of at least one extended release material and at least one release modifying material to form a second layer. When using a Wurster column, the nominal inlet air temperature is about 45° C. to about 55° C., preferably 48° C. to about 52° C., and the exhaust air temperature is about 28° C. to about 34° C., preferably 30° C. to about 32° C. The control release coating solution is prepared by first dissolving hydroxypropylmethyl cellulose (HPMC 6 cPs, Pharmacoat 606®) in Ethanol USP 95%, and then mixing the solution with ethylcellulose (Ethocel® 7 cPs) until dissolved, for example 45 minutes. The solution is constantly mixed during the coating step. Alternatively, the control release coating solution is prepared by mixing two separate solutions: a first solution of ethylcellulose (Ethocel® 7 cPs) dissolved in ethanol USP 95%, and a second solution of hydroxypropylmethyl cellulose (HPMC 6 cPs, Pharmacoat 606®) dissolved in purified water.

If necessary, after applying the second coating, the process may be followed by drying for a sufficient amount of time, for example 15 minutes. The drying may be performed by Wurster-drying at a nominal inlet air temperature of about 50° C. to about 55° C. until a nominal exhaust air temperature of about 40° C. is obtained.

Once the cores are coated twice, the coated units or multiparticulates are filled into capsules of a desired size. Capsules may be of size #2 or #4 for a 200 mg/dose or 100 mg/dose, respectively. In one embodiment, the capsule is a hard gelatin capsule with sufficient drug to have 2 mg or 4 mg of tolterodine L-tartrate.

The present invention encompasses a multiparticulate pharmaceutical composition of tolterodine having a reproducible dissolution profile comprising a combination of at least a first and a second population of multiparticulates having tolterodine and at least one pharmaceutically acceptable excipient, wherein the dissolution profile of a first batch or lot of the pharmaceutical composition that is measured at 4 hours differs by no more than 5% from the dissolution profile of other lots or batches of the pharmaceutical composition.

The multiparticulate pharmaceutical composition characteristics are as described above for the stable multiparticulate formulation.

Also provided is a method for preparing for preparing a multiparticulate pharmaceutical composition of tolterodine having a reproducible dissolution profile comprising: a) preparing at least two populations of multiparticulates having different dissolution profiles, wherein the difference is more than about 5% at 4 hours of dissolution, and more than about 10% at 12 hours of dissolution; b) characterizing the dissolution profiles of each population; and c) mixing a weighted portion of each population to obtain a pharmaceutical composition having a reproducible dissolution profile.

The present invention further encompasses a method of preparing a tolterodine formulation comprising combining a plurality of tolterodine containing multiparticulates into a dosage unit.

Having described the invention with reference to certain preferred embodiments, other embodiments will become apparent to one skilled in the art from consideration of the specification. The invention is further defined by reference to the following examples describing in detail the process and compositions of the invention. It will be apparent to those skilled in the art that many modifications, both to materials and methods, may be practiced without departing from the scope of the invention.

### EXAMPLES

### Examples 1-3

### Effect of Core Composition on Drug Release Profile

Cores were charged into a fluidized bed device equipped with a Wurster column (Wurster fluid bed) and coated with a drug containing dispersion at a nominal inlet air temperature of 50° C. to 55° C. and exhaust air temperature of 30° C. to 32° C. The drug containing dispersion was made of hydroxypropyl cellulose 100 cPs (1 mg/core) in purified water mixed with micronized tolterodine L-tartrate (4 mg/core) to form a homogenous dispersion. After coating, the coated core was dried (Wurster drying) at a nominal inlet air temperature of 55° C.

The coated cores were re-charged into the Wurster fluid bed and coated with control release coating at a nominal inlet air temperature of 50° C. to 52° C. and exhaust air temperature of 30° C. to 32° C. The control release coating was made of two solutions: (1) ethylcellulose 7 cPs (25.6 mg/core) in ethanol (USP 95%), and (2) hydroxypropylmethyl cellulose 6 cPs (6.4 mg/core) dissolved in purified water. After coating, the coated multiparticulate was dried (Wurster drying) at a nominal inlet air temperature of 55° C. until the nominal exhaust air temperature was about 40° C. Table 1 summarizes the composition of the multiparticulates of Examples 1-3.

TABLE 1
Formulation of multiparticulates of Examples
1, 2, and 3 by weight (mg).

| Component | Example 1 | Example 2 | Example 3 |
|---|---|---|---|
| Tolterodine L-tartrate | 4.0 | 4.0 | 4.0 |
| Sugar spheres, 25-30 mesh | 155.0 | | 93.0 |
| Microcrystalline cellulose spheres, | | 155.0 | 62.0 |
| 25-35 mesh | | | |
| Ethylcellulose 7 cPs (extended | 25.6 | 25.6 | 25.6 |
| release polymer) | | | |
| Hydroxypropylmethyl cellulose 6 cPs | 6.4 | 6.4 | 6.4 |
| (release modifying polymer) | | | |
| Hydroxypropyl cellulose 100 cPs | 1.0 | 1.0 | 1.0 |
| (binder) | | | |
| Total weight | 192.0 | 192.0 | 192.0 |

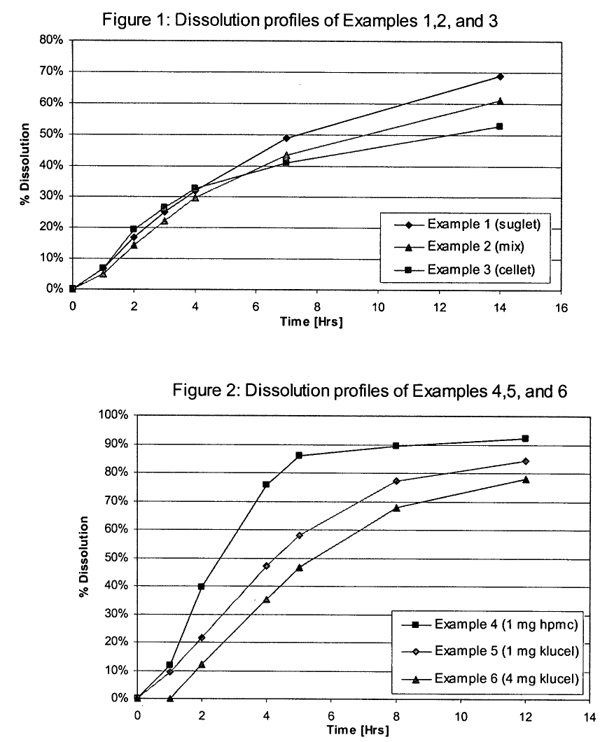

Multiparticulates of each formulation were dissolved at 37° C. in 0.05 M phosphate buffer with a pH 6.8 using a USP dissolution test apparatus I and using 900 ml of solution (when testing over 0-14 hours). The drug release profile or dissolution profile was measured at time 0, 2, 3, 4, 7, and 14 hours. Table 2 summarizes the dissolution data for each multiparticulate and FIG. 1 illustrates the drug dissolution profile for multiparticulates of Examples 1-3. The dissolution profiles are given in terms of percentages where 100% is equivalent to 4 mg of tolterodine tartrate.

TABLE 2
Dissolution Profile of Examples 1, 2, and 3.

| Time (Hrs) | Example 1 | Example 2 | Example 3 |
|---|---|---|---|
| 1 | 7% | 7% | 5% |
| 2 | 17% | 19% | 14% |
| 3 | 25% | 27% | 22% |
| 4 | 32% | 33% | 30% |
| 7 | 49% | 41% | 43% |
| 14 | 69% | 53% | 61% |

### Examples 4-6

### Effect of the Composition of the Drug Containing Layer

Using the methodology described in Example 1 three sets of coated multiparticulates were prepared. In Examples 4-6 the composition of the core was kept constant and the composition of the drug containing layer was modified. In particular, the quantity of the hydrophilic polymer binder was varied. The formulation for each multiparticulate is set forth in Table 3.

TABLE 3
Composition of Multiparticulates of Examples
4, 5, and 6 by weight (mg).

| Component | Example 4 | Example 5 | Example 6 |
|---|---|---|---|
| Tolterodine L-tartrate | 4.0 | 4.0 | 4.0 |
| Sugar spheres, 25-30 mesh | 93.0 | 93.0 | 93.0 |
| Microcrystalline cellulose spheres, | 62.0 | 62.0 | 62.0 |
| 25-35 mesh | | | |
| Ethylcellulose 7 cPs | 25.6 | 25.6 | 26.2 |
| Hydroxypropylmethyl cellulose 6 cPs | 6.4* | 7.4** | 6.6* |
| Hydroxypropyl cellulose 100 cPs | 1.0 | NA | 4.0 |
| Total weight | 192.0 | 192.0 | 195.8 |

*release modifying polymer in extended release layer
**6.4 mg release modifying polymer in extended release layer and 1 mg as binder in drug layer

Multiparticulates of each formulation were dissolved at 37° C. in 0.05 M Phosphate buffer with a pH 6.8 using a USP dissolution test apparatus I and using 800 ml of solution (when testing over 0-12 hours). The drug release profile was measured at time 0, 1, 2, 4, 5, 8, and 12 hours. Table 4 summarizes the data for each multiparticulate and FIG. 2 illustrates the drug dissolution profile for multiparticulates of Examples 4-6.

TABLE 4
Dissolution profile of Examples 4, 5, and 6

| Time (Hrs) | Example 4 | Example 5 | Example 6 |
|---|---|---|---|
| 1 | 9% | 12% | 0% |
| 2 | 22% | 40% | 12% |
| 4 | 47% | 76% | 35% |
| 5 | 58% | 86% | 47% |
| 8 | 77% | 89% | 68% |
| 12 | 84% | 92% | 78% |

As illustrated by the data of Table 4 and FIG. 2, the dissolution profile can be raised by selecting a hydrophilic polymer binder with relatively high water solubility, as illustrated in Examples 4 and 5. In contrast, the dissolution profile can be lowered by increasing the amount of hydrophilic polymer binder as illustrated in Example 6.

### Examples 7-9

### Effect of Ratio of Components in Control Release Layer for Hydroalcoholic ER Solution

Using the methodology described in Example 1 three sets of coated multiparticulates were prepared. In Examples 7-9, the composition of the core and drug containing layer was kept constant. The ratio between ethylcellulose (extended release polymer) and HPMC (release modifying polymer) in the second layer was systematically varied from 6:1 to 3:1, respectively. The formulation for each multiparticulate is set forth in Table 5.

TABLE 5
Formulation of Multiparticulates of Examples
7, 8, and 9 by weight (mg).

| Component | Example 7 | Example 8 | Example 9 |
|---|---|---|---|
| Tolterodine L-tartrate | 4.0 | 4.0 | 4.0 |
| Sugar spheres, 25-30 mesh | 93.0 | 93.0 | 93.0 |
| Microcrystalline cellulose spheres, | 62.0 | 62.0 | 62.0 |
| 25-35 mesh | | | |
| Ethylcellulose 7 cPs | 20.4 | 19.2 | 18.6 |
| Hydroxypropylmethyl cellulose 6 cPs | 3.6 | 4.8 | 5.4 |
| Hydroxypropyl cellulose 100 cPs | 1.0 | 1.0 | 1.0 |
| Total weight | 184.0 | 184.0 | 184.0 |

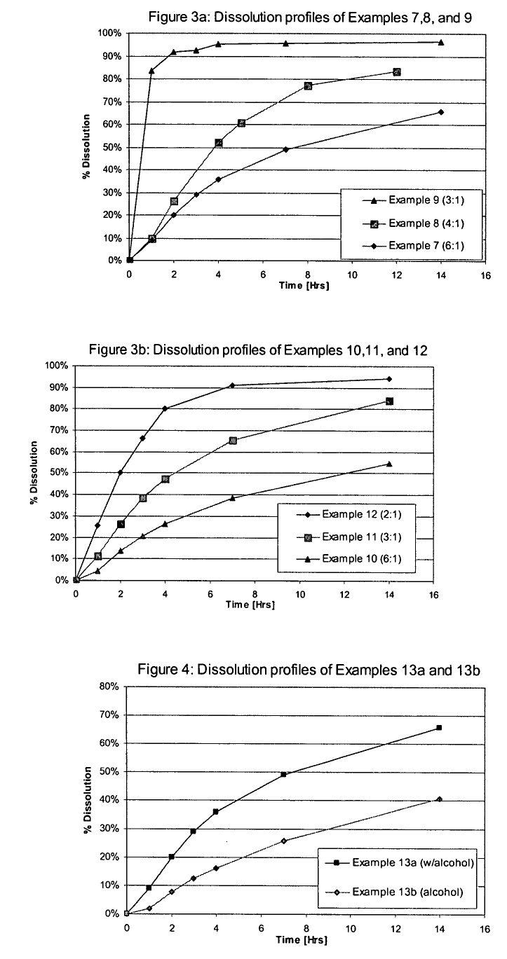

Multiparticulates of each formulation were dissolved at 37° C. in 0.05 M Phosphate buffer with a pH 6.8 using a USP dissolution test apparatus I and using 800 ml of solution (when testing over 0-12 hours). The drug release profile was measured at the times indicated below. Table 6 summarizes the data for each multiparticulate and FIG. 3a illustrates the drug dissolution profile for multiparticulates of Examples 7-9.

TABLE 6
Dissolution Profile of Examples 7, 8, and 9.

| Time | | Time | | |
|---|---|---|---|---|
| (Hrs) | Example 8 | (Hrs) | Example 7 | Example 9 |
| 1 | 10% | 1 | 9% | 84% |
| 2 | 27% | 2 | 20% | 92% |
| 4 | 52% | 3 | 29% | 93% |
| 5 | 61% | 4 | 36% | 95% |
| 8 | 78% | 7 | 49% | 96% |
| 12 | 84% | 14 | 66% | 96% |

### Examples 10-12

### Effect of Ratio of Components in Control Release Layer for Alcoholic ER Solution

Cores were charged into a fluidized bed device equipped with a Wurster column (Wurster fluid bed) and coated with a drug containing layer as detailed in Example 1. The coated cores were re-charged into the Wurster fluid bed and coated with a control release coating at a nominal inlet air temperature of 48° C. to 50° C. and exhaust air temperature of 32° C. to 34° C. The control release coating was made of ethylcellulose 7 cPs and hydroxypropylmethyl cellulose 6 cPs, both dissolved in ethanol (USP 95%). The hydroxypropylmethyl cellulose was dissolved first, while the ethylcellulose was added second, and the final solution was constantly mixed during the coating process. After coating, the coated multiparticulate was dried (Wurster drying) at a nominal inlet air temperature of 55° C. until the nominal exhaust air temperature was about 40° C. Table 7 summarizes the composition of the multiparticulates of Examples 10-12.

TABLE 7
Formulation of Multiparticulates of Examples
10, 11, and 12 by weight (mg).

| | Example | Example | Example |
|---|---|---|---|
| Component | 10 | 11 | 12 |
| Tolterodine L-tartrate | 4.0 | 4.0 | 4.0 |
| Sugar spheres, 25-30 mesh | 93.0 | 93.0 | 93.0 |
| Microcrystalline cellulose spheres, | 62.0 | 62.0 | 62.0 |
| 25-35 mesh | | | |
| Ethylcellulose 7 cPs | 13.6 | 12.0 | 10.4 |
| Hydroxypropylmethyl cellulose 6 cPs | 2.4 | 4.0 | 5.6 |
| Hydroxypropyl cellulose 100 cPs | 1.0 | 1.0 | 1.0 |
| Total weight | 176.0 | 176.0 | 176.0 |

Multiparticulates of each formulation were dissolved at 37° C. in 0.05 M Phosphate buffer with a pH 6.8 using a USP dissolution test apparatus I and using 900 ml of solution (when testing over 0-14 hours). The drug release profile was measured at the times indicated below. Table 8 summarizes the data for each multiparticulate and FIG. 3b illustrates the drug dissolution profile for multiparticulates of Examples 10-12.

TABLE 8
Dissolution profile of Examples 10, 11, and 12

| Time (Hrs) | Example 10 | Example 11 | Example 12 |
|---|---|---|---|
| 1 | 5% | 11% | 26% |
| 2 | 14% | 26% | 50% |
| 3 | 21% | 39% | 67% |
| 4 | 27% | 47% | 80% |
| 7 | 39% | 65% | 91% |
| 14 | 55% | 84% | 95% |

### Example 13

### Effect of Solvent Selection in Control Release Layer

Using the methodology described in Examples 1 and 10 two sets of coated multiparticulates were prepared. In Example 13, the composition of the core and drug containing layer was kept constant. Using the formulation of Example 7, the solvent for making the control release layer was varied. In Example 13a, the controlled release layer was made from a solution that was 7.3% by weight of a hydro-alcoholic solution, where the ratio of water to alcohol was 16:84 by weight. In Example 13b, the solution was 7.1% by weight alcoholic solution using 95% ethanol (USP).

Multiparticulates of each formulation were dissolved at 37° C. in 0.05 M Phosphate buffer with a pH 6.8 using a USP dissolution test apparatus I and using 900 ml of solution (when testing over 0-14 hours). The drug release profile was measured at the times indicated below. Table 9 summarizes the data for each multiparticulate and FIG. 4 illustrates the drug dissolution profile for multiparticulates of Examples 13a and 13b.

TABLE 9
Dissolution Profile of Examples 13a and 13b.

| Time (Hrs) | Example 13a | Example 13b |
|---|---|---|
| 1 | 9% | 2% |
| 2 | 20% | 8% |
| 3 | 29% | 13% |
| 4 | 36% | 16% |
| 7 | 49% | 26% |
| 14 | 66% | 41% |

### Example 14-17

### Effect of Polymer Viscosity in Extended Release Layer

Using the methodology described in Example 1 four sets of coated multiparticulates were prepared. In Examples 14-17, the composition of the core and drug containing layer was kept constant. The viscosity of each polymer in the control release layer was varied. In Examples 14-17, the viscosity of the ethylcellulose (extended release polymer) and/or HPMC (release modifying polymer) in the second layer was systematically varied. The formulation for each multiparticulate is set forth in Table 10.

TABLE 10
Formulation of Multiparticulates of Examples 14, 15, 16, and 17 by weight (mg)

| Component | Example 14 | Example 15 | Example 16 | Example 17 |
|---|---|---|---|---|
| Tolterodine L-tartrate | 4.0 | 4.0 | 4.0 | 4.0 |
| Sugar spheres, 25-30 mesh | 93.0 | 93.0 | 93.0 | 93.0 |
| Microcrystalline cellulose | 62.0 | 62.0 | 62.0 | 62.0 |
| spheres, 25-35 mesh | | | | |
| *Ethylcellulose 7 cPs | 25.6 | 25.6 | 25.6 | NA |
| *Ethylcellulose 50 cPs | NA | NA | NA | 25.6 |
| **HPMC 6 cPs | 6.4 | NA | 10.4*** | 10.4*** |
| **HPMC 3 cPs | NA | 6.4 | NA | NA |
| Hydroxypropyl Cellulose 100 cPs | 4.0 | 4.0 | NA | NA |
| Total weight | 195.0 | 195.0 | 195.0 | 195.0 |

*extended release polymer
**release modifier (hydroxypropylmethyl cellulose)
***4 mg of HPMC 6 cPs are also used as a binder in the first layer. 6.4 mg, serve as release modifier.

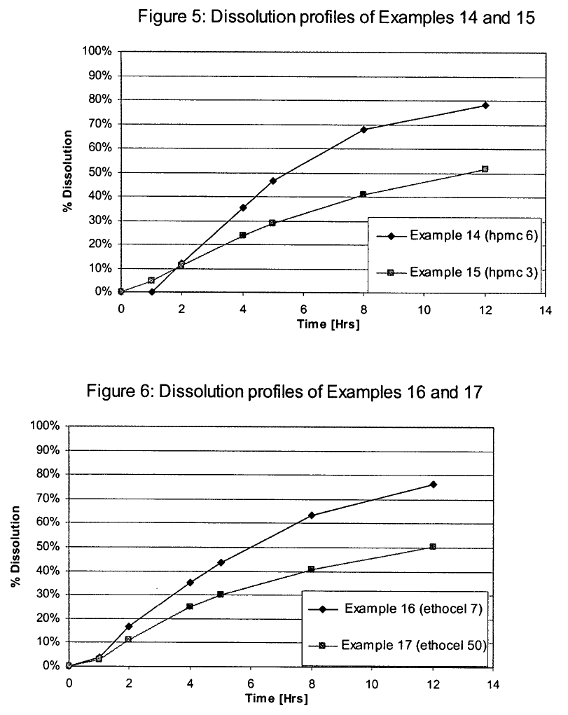

Multiparticulates of each formulation were dissolved at 37° C. in 0.05 M Phosphate buffer with a pH 6.8 using a USP dissolution test apparatus I and using 800 ml of solution (when testing over 0-12 hours). The drug release profile was measured at the times indicated below. Table 11 summarizes the data for each multiparticulate and FIGS. 5 and 6 illustrate the drug dissolution profile for multiparticulates of Examples 14, 15, 16, and 17.

TABLE 11
Dissolution Profile of Examples 14, 15, 16, and 17

| Time (Hrs) | Example 14 | Example 15 | Example 16 | Example 17 |
|---|---|---|---|---|
| 1 | 0% | 5% | 4% | 3% |
| 2 | 12% | 11% | 17% | 11% |
| 4 | 35% | 24% | 35% | 25% |
| 5 | 47% | 29% | 44% | 30% |
| 8 | 68% | 41% | 63% | 41% |
| 12 | 78% | 52% | 77% | 51% |

### Examples 18-20

### Effect of Core Materials on Drug Stability

Using the methodology described in Example 1, three sets of coated multiparticulates were prepared. The compositions of the drug containing layer and extended release layer were kept constant and the core material was varied. In Examples 18-20, cores of Suglets® or Cellets® were coated with a thin hydrophilic layer containing hydroxypropylmethyl cellulose and polyethylene glycol, followed by a drug containing layer of a hydroalcoholic solution using hydroxypropylmethyl cellulose as a binder and polyethylene glycol as an antitacking agent. A non-micronized drug substance was applied in the drug containing layer. The control release layer composed of ethylcellulose as an extended release polymer and polyethylene glycol as a plasticizer/release modifying polymer was then applied. The formulation of the multiparticulates of Examples 18, 19, and 20 are summarized in Table 12. In Example 20, the calculations were based on a 50:50 mixture of cores.

TABLE 12
Formulation of Multiparticulates of Examples 18, 19, and
20 by weight (mg).

| Component | Example 18 | Example 19 | Example 20* |
|---|---|---|---|
| Tolterodine L-tartrate | 4.0 | 4.0 | 4.0 |
| Sugar spheres, 18-20 mesh | 120.0 | NA | 60.0 |
| Microcrystalline cellulose | NA | 120.0 | 60.0 |
| spheres, 25-35 mesh | | | |
| Ethylcellulose 7 cPs | 25.7 | 27.1 | 26.4 |
| HPMC 6 cPs | 16.0 | 16.0 | 16.0 |
| Polyethylene Glycol 6000 | 6.0 | 6.0 | 6.0 |
| Polyethylene Glycol 400 | 9.4 | 9.4 | 9.4 |
| Total weight | 181.1 | 182.5 | 181.8 |

*Theoretical calculation based on a 50:50 w/w physical mixture.

The final coated pellets were stored in an air storage oven at 45° C. and 100% relative humidity (RH) for seven to ten days. Subsequently, the dissolution profile for each sample was compared with the dissolution profile of coated pellets stored at room temperature (RT). Table 13 summarizes the dissolution data for the multiparticulates.

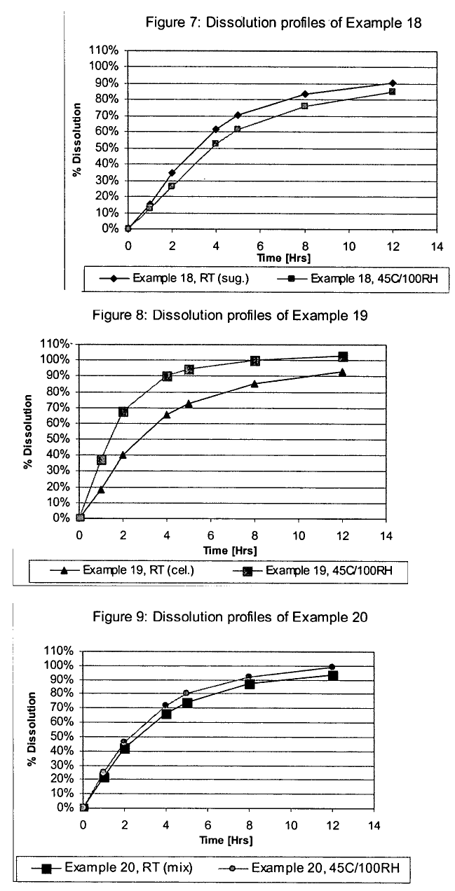

Example 20 was prepared by mixing equal amounts by weight of the two formulations described in Examples 18 and 19. The dissolution profile of the physical mixture at RT and at 45° C./100RH is illustrated in FIGS. 7-9. The ratio between the two different components can be slightly modified to obtain a stable dissolution profile.

Multiparticulates of each formulation, kept under either room temperature or 45° C. at 100% relative humidity, were dissolved at 37° C. in 0.05 M Phosphate buffer with a pH 6.8 using USP dissolution test apparatus I and using 800 ml of solution (when testing over 0-12 hours). The drug release profile was measured at the times indicated below. Table 13 summarizes the data for each multiparticulate and FIGS. 7, 8, and 9 illustrate the drug dissolution profile for multiparticulates of Examples 18, 19, and 20, respectively.

TABLE 13
Dissolution Profile for Examples 18, 19, and 20.

| Time | Example 18, | Example 18, | Example 19, | Example 19, | Example 20, | Example 20, |
|---|---|---|---|---|---|---|
| (Hrs) | RT | 45° C./100RH | RT | 45° C./100RH | RT | 45° C./100RH |
| 1 | 15% | 13% | 18% | 37% | 22% | 25% |
| 2 | 35% | 26% | 40% | 68% | 42% | 46% |
| 4 | 62% | 53% | 65% | 91% | 66% | 72% |
| 5 | 70% | 61% | 73% | 94% | 74% | 80% |
| 8 | 84% | 76% | 85% | 100% | 87% | 92% |
| 12 | 91% | 85% | 93% | 103% | 94% | 99% |

### Examples 21 and 22

### Effect of Drug Micronization on Drug Release Profiles

Using the methodology described in Example 1 two sets of coated multiparticulates were prepared. The composition of the drug containing layer was altered to either apply a drug solution or drug dispersion with hydroxypropylmethyl cellulose as a binder and polyethylene glycol as an antitacking agent. The control release layer was composed of ethylcellulose as an extended release polymer and polyethylene glycol as a plasticizer/release modifying polymer. In Examples 21 and 22, the core, Suglets®, was coated with either a drug solution (Example 21) or drug dispersion (Examples 22). The formulation of the multiparticulates of Examples 21 and 22 are summarized in Table 14.

TABLE 14
Formulation of Multiparticulates of Examples 21 and
22 by weight (mg).

| Component | Example 21 | Example 22 |
|---|---|---|
| Tolterodine L-tartrate, | 4.0 | NA |
| Tolterodine L-tartrate, Micron. | NA | 4.0 |
| Sugar spheres, 25-30 mesh | 121 | 144.0 |
| Ethylcellulose 7 cPs | 19.8 | 23.5 |
| Hydroxypropylmethyl Cellulose 6 cPs | 8.0 | 8.0 |
| Polyethylene Glycol 6000 | 2.0 | 2.0 |
| Polyethylene Glycol 400 | 7.2 | 8.1 |
| Total weight | 162.0 | 189.6 |

The final coated pellets were stored in an air storage oven at 45° C. and 100% relative humidity (RH) for seven to ten days. Subsequently, the dissolution profile for each sample was compared with the dissolution profile of coated pellets stored at room temperature (RT). Table 15 summarizes the dissolution data for the multiparticulates.

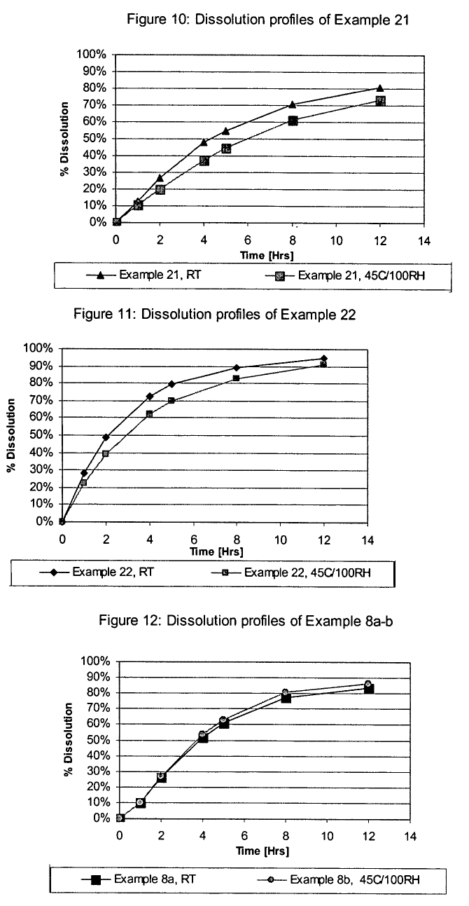

Multiparticulates of each formulation were dissolved at 37° C. in 0.05 M Phosphate buffer with a pH 6.8 using USP dissolution test apparatus I and using 800 ml of solution (when testing over 0-12 hours) under either room temperature or 45° C. at 100% relative humidity. The drug release profile was measured at the times indicated below. Table 15 summarizes the data for each multiparticulate and FIGS. 10 and 11 illustrate the drug dissolution profile for multiparticulates of Examples 21 and 22.

TABLE 15
Dissolution Profile for Examples 21 and 22.

| Time | Example 21, | Example 21, | Example 22, | Example 22, |
|---|---|---|---|---|
| (Hrs) | RT | 45° C./100RH | RT | 45° C./100RH |
| 1 | 13% | 11% | 28% | 22% |
| 2 | 26% | 20% | 49% | 39% |
| 4 | 48% | 37% | 73% | 62% |
| 5 | 55% | 45% | 79% | 70% |
| 8 | 70% | 61% | 89% | 83% |
| 12 | 81% | 74% | 95% | 91% |

Better drug stability is, however, achieved when the drug dispersion was applied on a combination of cores of sugar spheres, Suglets®, and microcrystalline cellulose spheres, Cellets®, such as those discussed in Example 8. The preferred ratio was 60:40 (Suglets®:Cellet®, respectively). The drug release profile for the multiparticulates of Example 8 was studied under either room temperature (Example 8a) or 45° C. at 100% relative humidity (Example 8b). The drug release profile was measured at the times indicated below. Table 16 summarizes the data for the multiparticulate of Examples 8a and 8b, and FIG. 12 illustrates the drug dissolution profile for each.

TABLE 16
Dissolution Profile for Examples 8a and 8b at RT and 45° C./100 RH.

| Time (Hrs) | Example 8a, RT | Example 8b, 45° C./100 RH |
|---|---|---|
| 1 | 10% | 10% |
| 2 | 27% | 27% |
| 4 | 52% | 54% |
| 5 | 61% | 63% |
| 8 | 78% | 81% |
| 12 | 84% | 87% |

### Example 23

### Controlled Release Formulations of Tolterodine Tartrate 2 mg and 4 mg

Sugar spheres, Suglets®, and microcrystalline cellulose spheres, Cellets®, were charged into a Glatt-Powder-Coater-Granulator (GPCG) 30 equipped with a Wurster column and coated at a nominal inlet air temperature of 50-55° C. and exhaust air temperature of 28-34° C. with a drug containing dispersion. The drug containing dispersion was prepared by dissolving hydroxypropyl cellulose (HPC, Klucel®) in purified water (ca. 15 min), and then mixing the solution with micronized Tolterodine L-tartrate for ca. 40 min to form a homogeneous dispersion. During the drug containing layer coating process, the homogeneous dispersion was continuously mixed. The coated spheres were dried by Wurster-drying for about 15 minutes at a nominal inlet air temperature of 60° C. until a nominal exhaust air temperature of 40° C. was obtained.

The multiparticulates coated with the drug containing layer were re-charged into the Wurster-equipped GPCG 30 and coated with a control release coating solution at a nominal inlet air temperature of 48-52° C. and exhaust air temperature of 30-34° C. The control release coating solution was prepared by first mixing hydroxypropylmethyl cellulose (HPMC 6 cPs, Pharmacoat 606 G®) in ethanol 95% (USP) for ca. 15 min, and then adding ethylcellulose (Ethocel® 7 cPs) to the solution and mixing for ca. 45 min. After application of the control release layer (under constant mixing) the multiparticulates were dried by Wurster-drying for 15 minutes at a nominal inlet air temperature of 50° C. until a nominal exhaust air temperature of 40° C. was obtained.

The coated multiparticulates were filled into size #2 (ca 200 mg/dose) or #4 (ca 100 mg/dose) hard gelatin capsules to obtain 4 mg and 2 mg dose of tolterodine L-tartrate, respectively. Table 17 summarizes the composition of the multiparticulates.

TABLE 17
Composition of Multiparticulates

| Material | Function | mg/capsule | mg/capsule* |
|---|---|---|---|
| Cores (spheres) | | | |
| Microcrystalline cellulose (500-710 | Swellable inert core | 64-74 | 69 |
| micron) | | | |
| Sugar spheres (600-710 micron) | Soluble inert core | 97-107 | 102 |
| Drug Containing Layer | | | |
| Tolterodine L-Tartrate Micronized | API | 4 | 4 |
| Klucel LF (hydroxypropyl cellulose) | Hydrophilic Polymer | 1-2 | 1 |
| | Binder | | |
| Purified Water | Process Solvent | NA | 45** |
| Control Release Coating | | | |
| Ethocel Premium 7 cps (ethylcellulose) | Extended release | 14-26 | 20 |
| | polymer | | |
| Hypromellose 2910 | Release Modifying | 4-10 | 7 |
| (hydroxypropylmethyl cellulose 6 cps) | polymer | | |
| Alcohol 95% | Process Solvent | NA** | NA** |

*preferred value.
**removed during process.

### Example 24

### Controlled Release Formulations of Tolterodine Tartrate 2 mg and 4 mg

Sugar spheres, Suglets®, and microcrystalline cellulose spheres, Cellets®, were charged into a Glatt-Powder-Coater-Granulator (GPCG) 30 equipped with a Wurster column and coated at a nominal inlet air temperature of 50-55° C. and exhaust air temperature of 28-34° C. with a drug containing dispersion. The drug containing dispersion was prepared by dissolving hydroxypropyl cellulose (HPC, Klucel®) in purified water (ca. 15 min), and then mixing the solution with micronized tolterodine L-tartrate for ca. 40 min to form a homogeneous dispersion. During the drug containing layer coating process, the homogeneous dispersion was continuously mixed. The coated cores were dried by Wurster-drying for about 15 minutes at a nominal inlet air temperature of 60° C. until a nominal exhaust air temperature of 40° C. was obtained.

The cores coated with the drug containing layer were re-charged into the Wurster-equipped GPCG 30 and coated with a control release coating material at a nominal inlet air temperature of 50-55° C. and exhaust air temperature of 30-34° C. The control release coating material was prepared by mixing two separate solutions of a first solution of ethylcellulose (Ethocel® 7 cPs) mixed with Ethanol 95% (USP) for about 40 minutes and a second solution of hydroxypropylmethyl cellulose (HPMC 6 cPs, Pharmacoat 606 G®) mixed with purified water for 15 minutes. The ethylcellulose solution was mixed with the hydroxypropylmethyl cellulose solution for ca. 15 min. After application of the control release layer the multiparticulates were dried by Wurster-drying for 15 minutes at a nominal inlet air temperature of 50° C. until a nominal exhaust air temperature of 40° C. was obtained.

The multiparticulates were filled into size #2 (ca 200 mg/dose) or #4 (ca 100 mg/dose) hard gelatin capsules to obtain 4 mg and 2 mg dose of tolterodine L-tartrate, respectively. Table 18 summarizes the compositions of the multiparticulates.

TABLE 18
Composition of Multiparticulates

| Material | Function | mg/capsule | mg/capsule* |
|---|---|---|---|
| Cores (spheres) | | | |
| Microcrystalline cellulose (500-710 | Swellable inert core | 67-57 | 62 |
| micron) | | | |
| Sugar spheres (600-710 micron) | Soluble inert core | 98-88 | 93 |
| Drug Containing Layer | | | |
| Tolterodine L-Tartrate Micronized | API | 4 | 4 |
| Klucel LF (hydroxypropyl cellulose) | Hydrophilic Polymer | 1-2 | 1 |
| | Binder | | |
| Purified Water | Process Solvent | NA | NA** |
| Control Release Layer | | | |
| Ethocel Premium 7 cps (ethylcellulose) | Extended release | 24-40 | 32 |
| | coating | | |
| Hypromellose 2910 | Release Modifying | 6-10 | 8 |
| (hydroxypropylmethyl cellulose 6 cps) | coating | | |
| Alcohol 95% | Process Solvent | NA** | NA** |
| Purified Water | Process Solvent | NA** | NA** |

*preferred value.
**removed during process

What is claimed is:

1. A stable multiparticulate pharmaceutical composition of tolterodine comprising at least one pharmaceutically acceptable excipient and at least two populations of multiparticulates each population having tolterodine or a salt thereof and the ratio of the populations is from 90:10 to 10:90 by weight, wherein after storage for 1 month at 40° C. and 75% relative humidity the difference between the dissolution profile at 4 hours is no more than about 5% when compared to the dissolution profile at the time of manufacture.

2. The stable multiparticulate pharmaceutical formulation according to claim 1, wherein the populations comprise a first population of multiparticulates having a water soluble sphere core and a second population of multiparticulates having a nonsoluble and/or swellable sphere core.

3. The stable multiparticulate pharmaceutical formulation according to claim 2, wherein in the first or second population of multiparticulates each particulate has a core with a drug containing layer and a control release layer;
wherein the drug containing layer surrounds the core and has i) tolterodine and/or a metabolite or pharmaceutically acceptable salt or salts thereof and ii) at least one hydrophilic polymer binder; and the control release layer surrounds the drug containing layer and has at least one extended release material and at least one release modifying material.

4. The stable multiparticulate pharmaceutical formulation according to claim 1, wherein the tolterodine salt is tolterodine L-tartrate.

5. The stable multiparticulate pharmaceutical formulation according to claim 3, further comprising a water soluble polymer coating between the core and the drug containing layer.

6. The stable multiparticulate pharmaceutical formulation according to claim 3, wherein the control release layer further comprises a plasticizer.

7. The stable multiparticulate pharmaceutical formulation according to claim 2, wherein the ratio of the population having a water soluble sphere core to the population having a nonsoluble and/or swellable sphere core is about 90:10 to 90:10 by weight.

8. The stable multiparticulate pharmaceutical formulation according to claim 2, wherein the ratio of the population having a water soluble sphere core to the population having a nonsoluble and/or swellable sphere core is about 20:80 to 80:20 by weight.

9. The stable multiparticulate pharmaceutical formulation according to claim 2, wherein the core is sphere shaped and has a diameter of about 0.3 mm to about 1 mm.

10. The stable multiparticulate pharmaceutical formulation according to claim 2, wherein the core is sphere shaped and has a diameter of about 0.4 mm to about 0.8 mm.

11. The stable multiparticulate pharmaceutical formulation according to claim 2, wherein the first population has a sugar sphere core and the second population has a cellulose sphere core.

12. The stable multiparticulate pharmaceutical formulation according to claim 11, wherein the ratio of sugar sphere cores to cellulose sphere core in a ratio of 1:1 to 2:1 by weight.

13. The stable multiparticulate pharmaceutical formulation according to claim 3, wherein the ratio of tolterodine to hydrophilic polymer binder is about 1:2 to 5:1 by weight.

14. The stable multiparticulate pharmaceutical formulation according to claim 1, wherein the tolterodine is micronized and has a particle size distribution wherein the d(0.9) value is less than or equal to about 80 microns.

15. The stable multiparticulate pharmaceutical formulation according to claim 1, wherein the tolterodine is micronized and has a particle size distribution wherein the d(0.9) value is less than or equal to about 50 microns.

16. The stable multiparticulate pharmaceutical formulation according to claim 3, wherein the hydrophilic polymer binder is polyvinyl pyrollidone, hydroxypropyl cellulose, or hydroxypropyl methyl cellulose.

17. The stable multiparticulate pharmaceutical formulation according to claim 3, wherein the drug containing layer comprises about 1% to about 10% by weight of the final particulate.

18. The stable multiparticulate pharmaceutical formulation according to claim 3, wherein the ratio of the extended release material to the release modifying material is from about 6:1 to about 1.5:1 by weight.

19. The stable multiparticulate pharmaceutical formulation according to claim 3, wherein the extended release material is ethylcellulose or polymethacrylate polymer.

20. The stable multiparticulate pharmaceutical formulation according to claim 3, wherein the release modifying material is low viscosity hydroxypropyl methylcellulose.

21. The tolterodine formulation according to claim 19, wherein the ethylcellulose has a viscosity of about 7 cPs to about 50 cPs.

22. The stable multiparticulate pharmaceutical formulation according to claim 20, wherein the hydroxypropyl methylcellulose has a viscosity of about 3 cPs to about 6 cPs.

23. The stable multiparticulate pharmaceutical formulation according to claim 3, wherein the control release layer further comprises a plasticizer.

24. The stable multiparticulate pharmaceutical formulation according to claim 23, wherein the ratio of the extended release material and the release modifying material to plasticizer is about 25:1 to 10:1 by weight.

25. The stable multiparticulate pharmaceutical formulation according to claim 3, wherein the control release layer comprises about 4% to about 30% by weight of the final multiparticulate.

26. The stable multiparticulate pharmaceutical formulation according to claim 3, wherein the control release layer comprises about 6% to about 25% by weight of the final multiparticulate.

27. A process for preparing a stable multiparticulate pharmaceutical composition of tolterodine comprising:
mixing at least one pharmaceutically acceptable excipient and at least two populations of multiparticulates, each population having tolterodine or a salt thereof and each population having an unstable tolterodine dissolution profile, wherein the ratio of the populations is from 90:10 to 10:90 by weight, to obtain a stable multiparticulate pharmaceutical composition.

28. The process for preparing a stable multiparticulate pharmaceutical formulation according to claim 27, wherein the populations comprise a first population of multiparticulates having a water soluble sphere core and a second population of multiparticulates having a nonsoluble and/or swellable sphere core.

29. The process for preparing a stable multiparticulate pharmaceutical formulation according to claim 27, wherein the multiparticulates are prepared by:
providing at least one core comprising a combination of water soluble sphere and nonsoluble and/or swellable spheres in a ratio of about 10:90 to about 90:10 by weight;
applying to the core a drug containing material in an amount sufficient to form a drug containing layer to form a core with a drug containing layer;
and applying to the drug containing layer a control release material in an amount sufficient to form a control release layer;
wherein the drug containing material comprises i) a drug which is at least one antimuscarinic antagonist and ii) at least one hydrophilic polymer binder; and the control release material comprises at least one extended release polymer and at least one release modifying polymer.

30. The process according to claim 29, wherein the first applying step comprises charging the core into a fluidized bed device equipped with a Wurster column and applying a coating of the drug containing material to form the drug containing layer.

31. The process according to claim 29, wherein the drug containing material is a dispersion prepared by dissolving the hydrophilic polymer binder in purified water to form a solution and then mixing the solution with tolterodine to form a homogeneous dispersion.

32. The process according to claim 29, further comprising a drying step after applying the drug containing material to the core.

33. The process according to claim 29, wherein the second applying step comprises charging the core with a drug containing layer into a Wurster fluid bed and applying a control release material of at least one extended release polymer and at least one release modifying polymer.

34. The process according to claim 29, wherein the control release material is prepared by mixing two separate solutions of a first solution of ethylcellulose dissolved in ethanol and a second solution of hydroxypropylmethyl cellulose dissolved in purified water.

35. The process according to claim 29, wherein the control release material is prepared by mixing hydroxypropyl methylcellulose and ethylcellulose in ethanol.

36. The process according to claim 29, further comprising a drying step after applying the control release material.

37. A multiparticulate pharmaceutical composition of tolterodine having a reproducible dissolution profile comprising a combination of a first and a second population of multiparticulates each having tolterodine and at least one pharmaceutically acceptable excipient, wherein the dissolution profile of a first batch or lot of the pharmaceutical composition population that is measured at 4 hours differs by no more than 5% from the dissolution profile of subsequent lots or batches of the pharmaceutical composition.

38. A process for preparing a multiparticulate pharmaceutical composition of tolterodine having a reproducible dissolution profile over 4 hours comprising:
a) preparing at least two populations of multiparticulates having different dissolution profiles, wherein the difference in dissolution profile is more than 5% at 4 hours of dissolution and more than 10% at 12 hours of dissolution;
b) characterizing the dissolution profiles of each population; and
c) mixing a weighted portion of each population to obtain a pharmaceutical composition having a reproducible dissolution profile.

39. A method of preparing a tolterodine formulation comprising combining a plurality of tolterodine containing multiparticulates into a dosage unit.
